(19) United States
(12) Patent Application Publication
(10) Pub. No.: US 2010/0145014 A1
(43) Pub. Date: Jun. 10, 2010

(54) SYSTEM AND PROCESS FOR THE SYNTHESIS OF POLYMERS

(76) Inventors: Alain LAURENT, Grenoble (FR); Philippe Sarra-Bournet, Aix En Provence (FR); Olivier Aude, Marseille (FR); Paul Morgavi, La Ciotat (FR); Bernard Mandrand, Villeurbanne (FR); Marc Cuzin, Corenc (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/634,204

(22) Filed: Dec. 9, 2009

Related U.S. Application Data
(62) Division of application No. 10/547,129, filed on Jun. 29, 2006, now abandoned.

(30) Foreign Application Priority Data
Feb. 28, 2003 (DE) 103 08 931.4

(51) Int. Cl. C07K 1/04; C07H 1/00

(52) U.S. Cl. 530/334; 536/25.3

(57) ABSTRACT

The present invention relates to an automated polymer synthesis apparatus for synthesizing a polymer chain onto a solid substrates by sequentially adding polymer building blocks as well as to a method for synthesizing polymers on solid substrates by sequentially reacting polymer building blocks with reactive groups. The invention further relates to a biochip comprising a solid substrate with reactive groups where biomolecules are attached to and the remaining reactive groups are transformed into chemically inert species.

The present invention relates in a first aspect to an automated polymer synthesis apparatus for synthesizing polymer chains onto solid substrates by sequentially adding polymer building blocks and in a second aspect to a method for synthesizing polymers on solid substrates by sequentially reacting polymer building blocks with reactive groups. Further, the invention relates to a biochip comprising a solid substrate with reactive groups whereby biomolecules are attached to the reactive groups.

Today, the need for systems useful for the synthesis of polymers, especially in the field of synthesizing oligonucleotide or polypeptide polymers is constantly increasing. For this purpose, different synthetic methodologies have been developed. By far the most important two synthetic methodologies are i) the photochemical synthesis and ii) the “classical” chemical synthesis of such polymers. Both methodologies comprise the transformation of at least one functional group of building blocks of the desired polymer by subsequently reacting these building blocks to form the polymer.

The functional groups (usually terminal OH groups) of the building blocks are temporarily protected by intermediate protecting groups which will cleave when treated with appropriate reagents. These protecting groups are usually either acid labile groups like for example DMT (dimethoxytrityl) and its derivatives or photolabile groups like for example NPPOC (2-nitrophenyl-propyloxycarbonyl).

Many efforts have been undertaken in order to vary these protecting groups. For example U.S. Pat. No. 6,222,030 describes the use of carbonate protected hydroxyl groups in a 3′-5′ oligonucleotide.

Both synthetic methodologies are applicable as in-situ or ex-situ syntheses. In-situ synthesis is often preferred because it allows the facile build-up of different or identical polymer chains directly on the respective substrate without the need of a subsequent fixation of the polymer chain(s) onto a substrate.

By far, most of the synthetic methodologies known in the art use a sophisticated set-up in order to precisely define the reaction site where the polymer chain has to be built up. This comprises inter alia the use of a plurality of masks or a multitude of micromirrors for photochemical synthetic methodologies.

The chemical synthetic methodology using classical “wet-chemistry” encounters problems caused by the use of strong bases which often degrade the material of the apparatus where the synthesis takes place.

One of the major drawbacks of the aforementioned methods is the nature of the substrate to be employed in these reactions. In most cases glass slides or silica and silicon materials have been used which comprise hydroxyl groups on the surface of the material where the first polymer building block can be attached by various means. In order to overcome the drawbacks of glass (rigidity, fragility etc.) it has been proposed to coat the glass substrate with functionalizable materials or to use plastic substrates:

U.S. Pat. No. 6,258,454 discloses low surface energy functionalized surfaces on solid glass supports (slides) by treating the glass slides having hydrophilic moieties on its surface with a derivatizing composition containing a mixture of silanes. A first silane provides the desired reduction in surface energy, while the second silane enables functionalization with molecular moieties of interest such as initial monomers to be used in the solid phase synthesis of oligomers.

U.S. Pat. No. 6,146,833 discloses reagents for the immobilization of biopolymers, processes for the preparation and their subsequent use in the immobilization of biopolymers for analytical and diagnostic purposes. The reagents disclosed therein include a solid support fabricated of a polymeric material having at least one surface with pendant acyl fluoride functionalities. The solid support comprises and is fabricated of polymeric materials including ethylene acrylic acid or ethylene methacrylic acid copolymers and active polypropylene. Biopolymers cannot be attached directly to the surface of the polymer support, because they require additional linker groups which are onerous to introduce.

A further problem encountered in the automated synthesis procedures known in the art is the “blooming” in synthesizing high-density arrays.

U.S. Pat. No. 6,184,347 discloses a wash reagent employed for the bulk washing of the surface of high-density polymer arrays to remove unreacted polymeric building blocks from cells of the array while at the same time reacting with the unreacted monomer in order to prevent reaction of the reacted monomer with functional groups on the surface of the HDA outside of the region of the surface to which the reactive monomer is applied. Therefore, blooming of the droplets applied to the surface of a high-density array is minimized. The reactive wash solution is preferably methanol.

Another alternative is the synthesis of the polymers in pores of a defined size: U.S. Pat. No. 6,277,334 discloses a chemical reaction apparatus, materials and methods for the automated efficient synthesis of chemical species and molecular libraries. Oligomers and molecular libraries are synthesized in pores of porous substrates. The reaction takes place in the micropores of the substrates.

Further, various devices and methods for the polymer synthesis using arrays have been proposed in the art.

U.S. Pat. No. 5,472,672 discloses a polymer synthesis apparatus for building a polymer chain including a head assembly having an array of nozzles where each nozzle is coupled to a reservoir of a liquid reagent and a base assembly having an array of reaction sites. Different transport mechanisms are required in order to arrange the substrate exactly below the nozzle with the reagent used for the specific polymer building block.

U.S. Pat. No. 5,474,796 provides an apparatus and methods for the manufacture of arrays of functionalized binding sites on a support surface, especially for the synthesis of oligonucleotides and polypeptides. Hydrophilic spots in Arrays are surrounded by hydrophobic regions on the surface of a substrate. The solution comprising the monomeric polymer building blocks is applied to the hydrophilic reactions sites and will not mix with adjacent reaction sites due to the hydrophobic environment.

U.S. Pat. No. 5,529,756 discloses further a polymer synthesis apparatus for building a polymer chain including a head assembly with an array of nozzles where each nozzle is coupled to a reservoir to liquid reagents and a base assembly having an array of reaction vessels. Various transport mechanisms of the substrate are required in order to place the substrate under the nozzle which contains the agent of choice. Further, the set-up is quite complicated because each single nozzle is coupled to a reservoir of the liquid reagent.

Further problems encountered upon evaporating a liquid reagent applied to the surface of a substrate are the subject of the disclosure of U.S. Pat. No. 6,177,558. The evaporation of a liquid reagent during solid-phase synthesis or on micro-scale synthesis is reduced by providing an open solid support surface including at least one binding site which is functionalized with a reactive chemical moiety. A substantially controlled and minute volume of liquid reagent solution is deposited onto the support surface. The reagent solution includes reactants contained in at least one relatively high boiling point solvent which is preferentially a polar, aprotic solvent having a boiling point of at least about 140° C. and is selected from the group consisting of dinitriles, glymes, diglymes, etc.

U.S. Pat. No. 6,419,883 proposes to use micro droplets of a solution comprising a solvent having a boiling point of 150° C. or above, a surface tension of 30 dynes/cm or above, and a viscosity of 0.015 g/cm/sec. Preferred solvents comprise for example N-methyl-2-pyrrolidone, propylene carbonate or γ-butyrolactone. Further, this US patent discloses an automated system capable of processing one or more substrates during the synthesis of oligomers comprising an inkjet print head for spraying a microdroplet comprising a chemical species on a substrate, a scanning transport for scanning the substrate adjacent to the print head to selectively deposit the microdroplets at specified sites, a flow cell for treating the substrate on which the microdroplet is deposited by exposing the substrates to one or more selected fluids, and a treating transport for moving the substrate between the print head and the flow cell for treatment in the flow cell whereby the treating transport and the scanning transport are different elements. The system according to this reference requires a quite sophisticated apparatus.

Still further unsolved problems encountered in the automated synthesis of polymers, especially biopolymers, are that reactions often only proceed when activators are added to the single polymeric building blocks. After addition of the activator to the polymeric building blocks, the mixture is stored in an external reservoir. However, the mixture has only a short and limited shelf-lifetime and degradation of the components occurs within a few minutes; therefore the mixture cannot be used for longer synthesis cycles and is wasted. Since the reagents employed are usually expensive, this waste of reagents increases the overall costs for the synthesis of these polymers. To avoid the decomposition of a premixed activator/building block solution, it has been proposed to spray one droplet comprising the polymer building block which is to be deposited on the reactive site and one droplet comprising the activator from two separate nozzles at the same time. This leads to an unnecessarily complicated set-up with different types of nozzles, tubes and controlling devices. Quite often, the reaction on the reaction site is incomplete, because the mixture of the two separate droplets comprising the activator and the building block directly on the reaction site remains incomplete and is not homogeneous.

The object of the present invention is therefore to provide a system for an automated polymer synthesis which has an easy to handle set-up and which does not cause a waste of reagents due to their degradation upon prolonged storage.

This problem is solved by the present invention by an automated polymer synthesis system for the synthesis of polymer chain on a solid substrate by sequentially adding polymer building blocks, the system comprising an inkjet print head with a plurality of nozzles for the controlled generation of microdroplets containing a polymer building block, transport means for moving a substrate in a position adjacent to the print head and further to a treatment unit for treating the substrate on which a microdroplet has been deposited with a fluid and a micro mixing vessel adjacent to the print head wherein the polymer building block is mixed with an activator immediately prior to the reaction and whereby the micro-mixing vessel contains only the quantity of building block to be reacted in one reaction step.

The micro mixing vessel which is arranged adjacent to the print head offers the surprising advantage that only the exact quantity of a polymer building block to be reacted is mixed with an activator immediately prior to reaction and is completely used up during the reaction. Therefore, the solution comprising the activator mixed with the polymer building block, is not wasted after completion of the reaction and a degradation of the reagents is avoided. In a further especially preferred embodiment of the invention, the system comprises one micro mixing vessel for each single building block which enables the generation of only the exact amount of reagent required for each different building block at each step during the stepwise polymer synthesis and further to avoid traces of impurities upon change of the building block.

The term “adjacent” as used herein means that the micro mixing vessel is either in spatial vicinity to the ink-jet print head and linked e.g. via tubes to the ink-jet print head or, the micro mixing vessel is a part or an integral part of the ink-jet print head and may be separated from the head or from the nozzles via solid phase filters frits, diaphragms and the like.

The term “one reaction step” denotes the addition of at least one polymer building block, preferably in solution with an activator to the reaction site(s) where one step in the stepwise synthesis of the respective polymer by using the polymer building block is carried out.

In an especially preferred embodiment all of the elements of the system are arranged in a linear manner (one dimension) so that the transport means can transport the substrate only in one direction. This allows for the deposition of a plurality of microdroplets without complicated direction controlling means.

The mixing of the polymer building block with an activator is generally achieved by simply adding both components (usually in solution) to the micro mixing vessel with or without stirring.

It is understood that also a plurality of ink-jet print heads can be used within the scope of the invention.

A further surprising advantage of the invention is that only one type of transport means for moving a substrate is required in order to deposit a plurality of microdroplets in every direction on a substrate.

In a further preferred embodiment, the plurality of nozzles are linearly arranged on the ink-jet print-head. The linear arrangement provides an easy deposition of a plurality of microdroplets in one line without requiring further controlling means. Further, the ink-jet print-head has to be moved only in one direction to generate a huge number of microdroplets at the same time.

In a further preferred embodiment of the invention, each nozzle of the ink-jet print-head is selectively addressable. The generation of a plurality of polymer differing in the number and nature of their building blocks is therefore made considerably easier than with systems in prior art. (a building block may also be termed as “synthon”) It is further preferred that the inkjet print-head comprises a reservoir which is in fluid connection with each nozzle of the plurality of nozzles. This simplifies the system set-up because in prior art each nozzle has to be connected with a reservoir thus requiring more sophisticated controlling means and set-ups. As already explained in the foregoing, the micro mixing vessel according to the invention contains only the quantity of liquid comprising an activator and the polymer building block which is to be used in each reaction step. This allows for the use of only the exact amount needed for performing the chemical reaction.

It is preferred that each reservoir comprising one polymer building block is connected to a separate ink-jet print head to avoid contamination of the ink-jet print heads.

In an especially preferred embodiment all of the elements of the system are arranged in a rotating drum. This rotating arrangement requires only the displacement in one direction of the substrate(s) enabling repetitive easy-to-control passages of the substrate arranged below or above the ink-jet print head(s) without acceleration or slowing down. Further, this arrangement enables the easy addition at any desired time of further units (for example modules for additional polymers building blocks, like additional ink-jet print heads etc.) which is much more complicated when using a linear arrangement of the elements of the system.

Usually, the synthesis of polymers, especially biopolymers takes place on an array of previously predefined discrete regions (spots, locations) on a substrate. These discrete regions are isolated from each other and may be established by etching, barrier formation, masking and the like or by depositing reagents on the surface. However, all systems and processes in prior art require the generation of the discrete isolated regions before starting the first reaction step in the step-wise in-situ synthesis of the biopolymers at these discrete regions. This is a major drawback because the geometry of the discrete regions has to be designed according to the geometry of the arrangement of the nozzles at the ink-jet print head, which is tedious and ineffective and restricts severely the variation in the design of the array.

It has therefore been a further object of the invention to provide a method for the synthesis of polymers on a non-structured surface of a substrate which allows a customized generation of an array of reaction sites with a variable array geometry.

This further objective is solved by a method for synthesizing polymers on a solid substrate with a reactive surface by sequentially reacting polymer building blocks comprising reactive groups wherein at least one reactive group is protected with a removable protecting groups comprising the steps of
a) providing a polymeric surface whereby the polymeric surface comprises functional groups;
b) applying a first microdroplet comprising a first polymer building block on said surface;
c) reacting the first polymer building block with a functional group of the polymeric surface thereby attaching the first polymer building block to the polymeric surface;
d) removing a protecting group of the attached polymer building block;
e) applying onto said first microdroplet a second microdroplet comprising a second polymer building block, which is different from or the same as the first polymer building block;
f) reacting said second polymer building block with the de-protected reactive group of the first polymer building block;
g) if necessary sequentially repeating steps d)-f)
   and whereby after step c) the unreacted functional groups of the polymeric surface are transformed to a chemically inert species.

The method according to the invention provides the spatial definition and thereby the generation of an array of specific reaction sites by the reaction of a first microdroplet deposited on said functionalized surface whereby microspots are formed. In other words: the size of the first microdroplet defines the spatially limited reaction site (spot) where the generation of the polymer chain will take place. The transformation of the remaining reactive surface groups into chemically inert species (irreversible “surface capping”) allows to generate a specific reaction site pattern (array) with separated and distinct spots. It is one of the advantages of the method according to the invention, that even on an fully unstructured “empty” surface of a substrate, an array with a deliberately selectable geometry is generated without the need of structuring the surface before starting an in-situ synthesis on the prestructured reaction sites. The spots generated by the method according to the invention are discrete locations, separated by chemically inert, preferably also mechanically inert regions. However, also geometrically prestructured surfaces without predefined arrays of “spots” like 96-well microliter plates and the like can be used within the method of the present invention.

The term “chemically inert” means, that the species will not undergo a chemical reaction upon exposing the species to other chemical agents, solutions, exposure to electromagnetic irradiation and temperature. In an especially preferred embodiment the chemically inert species is also mechanically inert against scratching, etc.

The term “polymer” or “polymeric surface” in the context of the present invention comprises the presence of organic and inorganic polymer species which form the surface of a substrate. It is understood that glass (Si0₂) is also comprised within the term inorganic polymer. Further examples of inorganic polymers comprise polymerized organosilicon compounds, silicon-nitrogen compounds and the like. It is understood, that the substrate may be entirely made of a polymer, or that the substrate is made by a material distinct from its polymeric surface, which might be applied as a layer, coating and the like or even made within the substrate manufacturing process.

The next microdroplets comprising the second, third etc. polymer building block applied to the reaction sites react without the risk of blooming out only on the reaction site defined in the first reaction step. It is preferred that the first microdroplet has a diameter of 10 μm to 300 μm. It is understood that the second polymer building block (and all further polymer building blocks) may be the same or different.

It is especially preferred that the diameter of the first microdroplet is smaller than the diameter of the second microdroplet thereby enabling a complete reaction of the second microdroplet comprising a second polymer building block with the first polymer building block comprised within the first microdroplet. It is further preferred that the consecutive droplets, i.e. the third, fourth and so on have all the size of the second droplet. The difference in size between the first and the second etc. droplets has the advantage that mechanical imprecisions of the transport means of the substrate or of the controlling means which control the displacement of the print-head(s), which would lead to incomplete reaction at the reaction sites have no or at least no measurable effect on the result and the yield of each reaction step.

In a further preferred embodiment of the invention, the chemically inert species obtained after reacting an appropriate agent with the unreacted surface functional groups contains phosphorous. It is especially preferred that the phosphorous containing agent forms a phosphate group upon reaction with the reactive functional groups on the surface. The thus obtained reaction product forms an inert, especially chemically inert, surface between the reaction sites (spots) which are defined by the location of the first microdroplet. In another preferred embodiment, perfluorated phosphorous derivatives are used which create inert and hydrophobic zones around the reaction sites. The hydrophobicity has the advantageous effect, that the second etc droplets will be centered on the non hydrophobic portion, i.e. the spots of the substrate. Also, a blooming out is efficiently avoided.

It is preferred that the polymeric surface forms an integral part of the substrate, but it is also possible that for example a polymer film is coated on a substrate of another material, like another polymer, silicon, doped silicon, silicon nitride, etc.

In another preferred embodiment the polymeric surface consists of an organic polymer. Organic polymers like polyolefins, polyurethanes, polyacrylates, polyimides, polyesters and the like are easy to handle and to manipulate. Further, they can be specifically selected according to their chemical and physical properties so as to provide chemically inert polymer materials.

In an especially preferred embodiment the organic polymer surface comprises reactive groups selected from hydroxyl groups, amino groups, NRH groups and thiol groups. These groups allow an easy reaction with a polymer building block by creating a chemical bond, especially a covalent bond between the reactive group and the polymer building block.

Preferably, the polymer building blocks are nucleosides, nucleotides, for example oligonucleotides comprising up to 20 nucleosides or amino acids or oligopeptides or carbohydrate moieties. The method according to the invention can therefore successfully employed in the synthesis of a large number of different polymers, especially biopolymers.

In the method according to the invention it is especially preferred prior to step b) to mix an activator such as tetrazole, methyl- or ethylthiotetrazole and the like with a first polymer building block. But any other activator essentially known by a person skilled in the art may also be used. The activator allows for a faster and better (more complete) reaction between the two polymer building blocks for building up the polymer chain.

The problem underlying the present invention is further solved by a biochip comprising a a solid substrate with reactive groups on its polymeric surface and biomolecules attached by a chemical bond between the reactive groups and the biomolecules whereby the remaining reactive groups of the surface of the substrate not having reacted with a biomolecule have been transformed in chemically inert species. The biochip is obtainable by the process according to the invention and comprises usually 96 wells, each with an array comprising 128 microspots with oligonucleotide chains. The number of the arrays and of the microspots may vary according to the specific requirements and the above mentioned numbers are not meant to be limiting.

It is especially preferred that the chemically inert species and/or the agent for transforming the reactive functional groups of the surface in a chemically inert species comprise phosphorous or nitrogen compounds which are able to generate a chemically inert surface made of, e.g. phosphorous oxides, perfluorated phosphorous compounds and the like upon reaction or after a treatment (sintering, UV hardening etc) after reaction. In the case of nitrogen, it is preferred that the nitrogen containing moiety is already comprised on the surface of the substrate and requires only a chemical transformation like for example the transformation from an amine to an amide etc. The inert surface may also be generated by a further reaction as for example oxidation of Phosphorous (III) to Phosphorous (V) compounds, transformation of an amine to an inert amide and the like.

Preferably, the biomolecules are oligonucleotide sequences or polypeptide sequences or carbohydrate sequences which are able to conduct a variety of chemical reactions on a surface.

In an especially preferred embodiment, the biomolecules are oligonucleotides like RNA, DNA, LNA, and chimeras thereof.

It is further preferred that the substrate is an organic polymer comprising activated groups like OH, NRH, SH and the like so as to provide a plurality of attachment sites for various polymer building blocks with different reactive groups without requiring the introduction of specific linker moieties. The activation is achieved via different mechanisms essentially known to a person skilled in the art, for example via plasma treatment, laser treatment and the like.

It is especially preferred that the organic polymer is polypropylene which is chemically resistant to many or most of the known chemical reactions encountered in the synthesis of biological polymeric molecules.

### DEFINITIONS AND ABBREVIATIONS

Polymer building block: The term polymer building block (or “synthon”) denotes a chemical moiety which is comprised within the final polymer. The chemical moiety may therefore comprise functional groups before incorporation in the final polymer. Non limiting examples of suitable polymeric building blocks according to the invention are substituted or non-substituted phosphoramidites, mono-, oligo- and polynucleotides, amino acids, peptides, sugars (furanoses, riboses, etc.), biotin, avidin, streptavidin, antibodies and the like.

Microdroplet: A microdroplet of a solution comprises a high surface tension solvent with a boiling point of more than 150° C., preferably more than 220° C. and a surface tension of about 26-47 dyne/cm, preferably 30-39 dyne/cm, with a viscosity of 3.3-72 cP, preferably of 8-20 cP. Each microdroplet is a separate and discrete unit preferably having a volume of about 100 to 200 pL, most preferably between 5 pL and 70 pL. It is understood that the term “solution” as used herein comprises the solvent per se and the solute or several solutes.

The microdroplets when reacted to the surface form so-called spots or microdots. The arrays of polymers obtained according to the present invention are arranged in these microdots or spots which are separate and discrete units. The diameter of each microdot can be greater than 1,000 μm but ranges typically from about 5 μm-800 μm, preferably from about 10 μm-about 500 μm and most preferred from 20-200 μm.

The distance between the individual microdots is typically from about 1 μm-about 500 μm, preferably from about 20 μm-about 400 μm. Generally, the distance between the microdots should preferably be in the range of the respective site of the microdroplets to as to avoid a using of neighbouring spots.

The physical separation of the microspots is obtained by the reaction of the remaining functional groups with a non-removable protecting group preferably comprising phosphorous. These areas provide then an unreactive protective surface which is chemically inert.

The term “biomolecule” or “biopolymer” as used herein means any biological molecule in the form of a polymer, such as oligonucleotides, amino acids, peptides, proteins, carbohydrates, antibodies, etc. The term “nucleotide” as used herein comprises both deoxyribonucleosides and ribonucleosides. The term “oligonucleotide” refers to an oligonucleotide which has deoxyribonucleotide or ribonucleotide units.

Suitable nucleotides useful for the synthesis of oligonucleotides according to the present invention are those nucleotides that contain activated phosphorous containing groups such as phosphotriester, H-phosphonate and phosphoramidite groups.

The term “activator” usually means a catalyst which in the case of oligonucleotide synthesis is a catalyst which fosters the reaction between the 3′ phosphoramidite group of a nucleoside and the hydroxyl groups of the next nucleoside or nucleotide. This may be 5-methylthiotetrazole, tetrazole, or 5-ethylthiotetrazole, DCI or pyridiniumchloride.

The term “alkyl” as used herein refers to any saturated straight chain, branched or cyclic hydrocarbon group of 1 to 10 carbon atoms such as methyl, ethyl, n-propyl, isopropyl, n-butyl, etc. The term alkyl also includes the cycloalkyl groups such as cyclopentyl, cyclohexyl, cycloheptyl, etc.

The term “lower alkyl” denotes an alkyl group of 1 to 4 carbon atoms and includes methyl, ethyl, n-propyl, isopropyl, n-butyl, isobutyl, tbutyl.

The term “aryl” as used herein refers to any aromatic compound containing one to five aromatic rings either fused or linked and either unsubstituted or substituted with at least one substituent which are usually selected from the group consisting of amino, halogen, cyanide and lower alkyl. Preferred aryl substituents contain one to three fused aromatic rings. Aromatic compounds as used herein may or may not be heterocyclic, i.e. they might contain at least one heteroatom such as sulfur, nitrogen, phosphorous and the like.

The term “aralkyl” denotes a chemical compound containing both alkyl and aryl species, typically containing fewer than twenty carbon atoms. The term “aralkyl” is usually used to denote aryl-substituted alkyl groups.

The term “heterocyclic” refers to any five-membered or six-membered monocyclic structures or to an eight-membered to eleven-membered bicyclic structure which is either saturated or unsaturated. The heterocyclics comprise at least one heteroatom selected from the group consisting of nitrogen, oxygen, sulfur, phosphorous, arsenic and the like. The terms “nitrogen heteroatoms” and “sulfur heteroatoms” as well as “phosphorous heteroatoms” include any oxidized form of nitrogen, sulfur and phosphorous as well as a quarternized form of any basic nitrogen. Examples of “heterocyclic compounds” include piperidinyl, morpholinyl and pyrrolidinyl.

The term “halogen” is used in its usual sense to designate a chlorine, bromine, fluorine or iodine atom.

The term “oligonucleotide” as used herein designates polydeoxynucleotides (containing 2-deoxy-D-ribose), polyribonucleotides (containing D-ribose) to any other type of polynucleotide which is an N-glycoside of a purine or pyrimidine base and to other polymers containing non-nucleotidic backbones providing that the polymers contain nucleobases in a configuration which allows for base pairing and base stacking, such as is found in DNA and RNA.

By the term “protecting group” as used herein, a species is designated which prevents a segment of a molecule from undergoing a specific chemical reaction but which is removable from the molecule following completion of this reaction. The method of the present invention may also be used to synthesize peptides by standard solid phase peptide synthesis methodologies.

Typically, solid phase peptide synthesis is performed in a C to N direction. Thus, anchoring linkers are required such that cleavage at the end of the synthetic regime produces a C-terminal acid or amide. In preferred embodiments, a linker containing an activated carboxyl group is keyed to amino groups which can link to the activated surface of a support according to the invention. Any of the usual “temporary” protecting groups routinely used in polypeptide synthetic chemistry are suitable for use in the present invention. Non-limiting examples among these are, for example, BOC (t-butoxycarbonyl) and FMOC (N^{α9}-fluorenylmethyloxycarbonyl) groups. Other suitable amino protecting groups include but are not limited to 2-(4-biphenyl)propyl[2]oxycarbonyl (Bpoc), 1-(1-adamantyl)-1-methylethoxy-carbonyl (Adpoc) and the like. Representative activators or so-called in-situ coupling reagent suitable for use in the present invention include but are not limited to N,N′-dicyclohexylcarbodiimide (DCC) and the like. Preferred is their use in conjunction with the use of further accelerators or additives such as 1-hydroxybenzotriazole (HOBO, benzotriazol-1-yl-oxy-tris (dimethylamino)phosphonium hexafluorophosphate (BOP) and the like.

The method of the present of the present invention is advantageously employed to synthesize deoxyribonucleic acid (DNA) or ribonucleic acid (RNA) polymer species (so-called oligonucleotides) by any of the several known chemistries for solid-phase DNA or RNA synthesis including phosphite triester, phosphoramidite synthesis and H-phosphonate synthesis.

In principle, the method of the present invention is useful for practising any iterative nucleic acid synthetic technique. In an especially preferred embodiment of the invention, oligonucleotides are synthesized by the phosphoramidite method. For phosphoramidite synthesis according to the invention, the reactive sites on the surface of the substrate are sometimes functionalized with an additional spacer according to methods known in the art. The introduction of the spacer can take place before starting the generation of the polymer chain, or, for example after step a) of the process according to the invention. The spacer groups may also be applied to preselected portions of the reaction support only.

Typically, monomeric nucleotide or nucleoside polymer building blocks (also called synthons) have temporary protecting groups at appropriate nucleobase or 2′-O positions.

Solid phase nucleic acid synthetic techniques employ so-called temporary and permanent protecting groups in analogous fashion to solid phase peptide synthesis. Base labile protecting groups are used to protect the exocyclic amino groups of the heterocyclic nucleobases during the synthesis. This type of protection is usually achieved by acylation with acylating agents such as benzoylchloride and isobutyrylchloride. Acid labile protecting groups are used to protect the nucleotide 5′ hydroxyl during synthesis. Representative hydroxyl protecting groups are known to persons skilled in the art. These include but are not limited to dimethoxytrityl, monomethoxytrityl, trityl and 9-phenyl-xanthene (pixyl) groups. Dimethoxytrityl (DMT) protecting groups are widely used to the great acid lability which affords efficient removal even by very dilute acids.

The first step in the iterative chain elongation cycle according to the phosphoramidite technique is the removal of the 5′-O-protecting group (deprotection) of the initial monomer by immersing the reaction support in a solution of the deprotecting agent. This is followed by the addition of a rinsing reagent. Suitable reagents for deprotection include Lewis acids such as ZnBr₂, AlCl₃, BF₃ and TiCl₄ in various solvents such as dichloromethane nitromethane, tetrahydrofuran and mixed solvents such as nitromethane and lower alkyl alcohols such as methanol or ethanol and mixtures thereof. Protic acids, alone or in combinations, such as acetic acid, dichloroacetic acid, trichloroacetic acid, trifluoroacetic acid and toluenesulfonic acid may also be used.

Chains are lengthened by addition and reaction of activated 5′-O-protected monomeric synthons. In the phosphoramidite technique, a 5′-DMTr-deoxynucleoside-3′-O-(N,N-diisopropylamino)-β-cyanoethylphospite is deposited onto the reaction support. Phosphoramidites of numerous nucleosides are commercially available.

A mild organic catalyst or activator, typically tetrazole, ethylthiotetrazole or methylthiotetrazole is deposited onto the reaction support with the phosphoramidite. The coupling reaction is followed by the addition of a rinsing solvent, typically anhydrous acetonitrile.

After rinsing, a capping reagent is added for example by immersing the substrate in a solution of the capping reagent onto the preselected portions of the reaction support to cap free hydroxyl species remaining due to incomplete reaction of phosphite monomers. The capping reagent for, the “capping” of the non reacted hydroxyl groups of the nucleotide/nucleoside, which is typically a solution of an acid anhydride, also functions to reverse any inadvertent phosphitylation of guanine O-6 positions.

However, in the context of the present invention it has to be noted that there is a fundamental difference between the transformation of the non-reacted reactive groups on the surface of the solid support into a non-removable chemical inert species (also termed for the ease of the description as “surface capping”) and of the capping of non reacted hydroxyl groups of the nucleotide/nucleoside itself as described in the foregoing section.

Oxidation of the resulting phospite triester to the corresponding phosphate triester may be accomplished by adding an oxidant known in the art to be suitable such as a solution of alkaline iodine in water.

A person skilled in the art is fully aware that the synthesis of oligonucleotides may either take place in 3′-5′ or in 5′-3′ direction.

The method of the present invention may be employed in the synthesis of oligonucleotides having the naturally occurring nucleobases adenine (A), thymine (T), guanine (G), cytosine (C) and uracil (U) as well as non-naturally occurring nucleobases. Non-naturally occurring nucleobases are molecular moieties which are known in the art to mimic the function of naturally occurring nucleobases in their biological role as components of nucleic acids.

Oligonucleotide species having a wide variety of modifications to nucleobases, sugars or inter-sugar linkages can be prepared in accordance with the method of the invention which are generally applicable to the synthesis of any oligomers synthesizable by solid phase techniques as, for example, also to polycarbohydrates.

For example, the method of the present invention may be employed in the synthesis of S-phosphorodithioates, phosphorothioates, etc.

The polymers produced according to the method of the invention may be composed of more than one type of monomeric subunit (for example, amino acids, peptide nucleic acids, nucleotides, sugars (carbohydrates), etc.) and may possess more than one type of inter-subunit linkage. Illustrative polymers produced according to the method of the invention include peptides, peptoids (N-alkylated glycines), α-polyesters, polythioamides, N-hydroxy amino acids, β-esters, polysulfonamides, N-alkylates polysulfonamides, polyureas, peptide nucleic acids, nucleotides, polysaccharides, polycarbonates, oligonucleotides, oligonucleosides and the like and chimeric molecules that contain one or more of these polymers joined together as a single macro molecule.

Libraries of monomeric species can also be prepared by the method of the invention. These include benzodiazepine libraries and other such analog libraries including but not limited to antihypertensive agents, antiulcer drugs, antifungal agents, antibiotics, antiinflammatories, etc.

A person skilled in the art is fully aware that the scope of the invention does not only comprise the above-mentioned features alone but also combinations thereof and that the scope of the invention also comprises every single feature in relation to the invention as described herein.

The invention is further described in detail in the following description of preferred embodiments with respect to the figures.

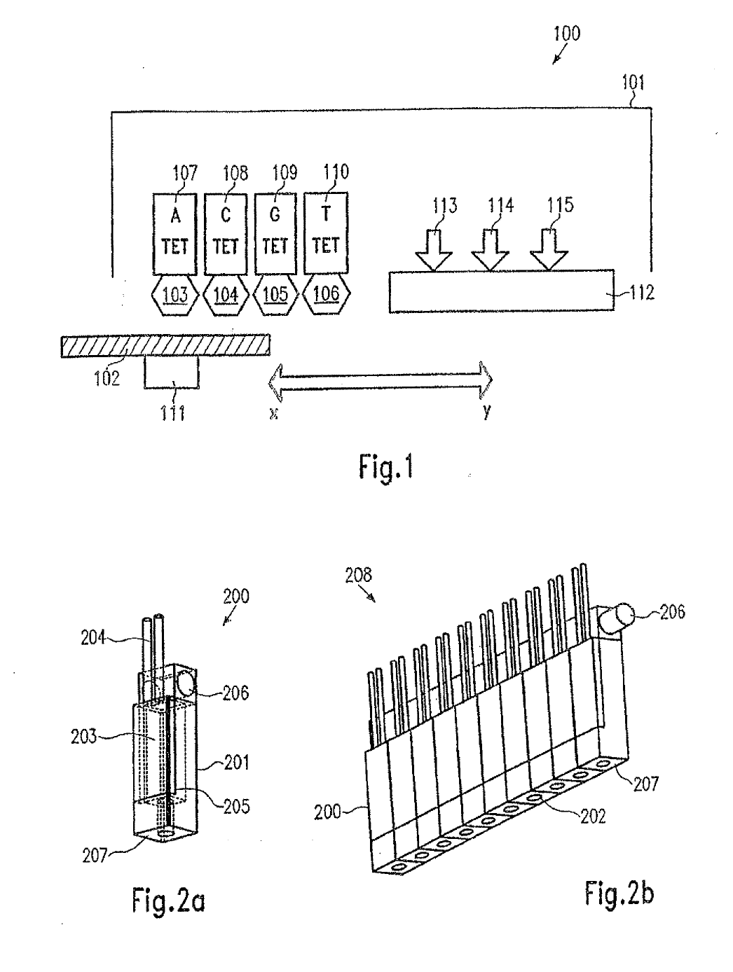

FIG. 1 schematically depicts a preferred embodiment of a system according to the invention;

FIG. 2 shows a schematic inkjet print head according to the invention;

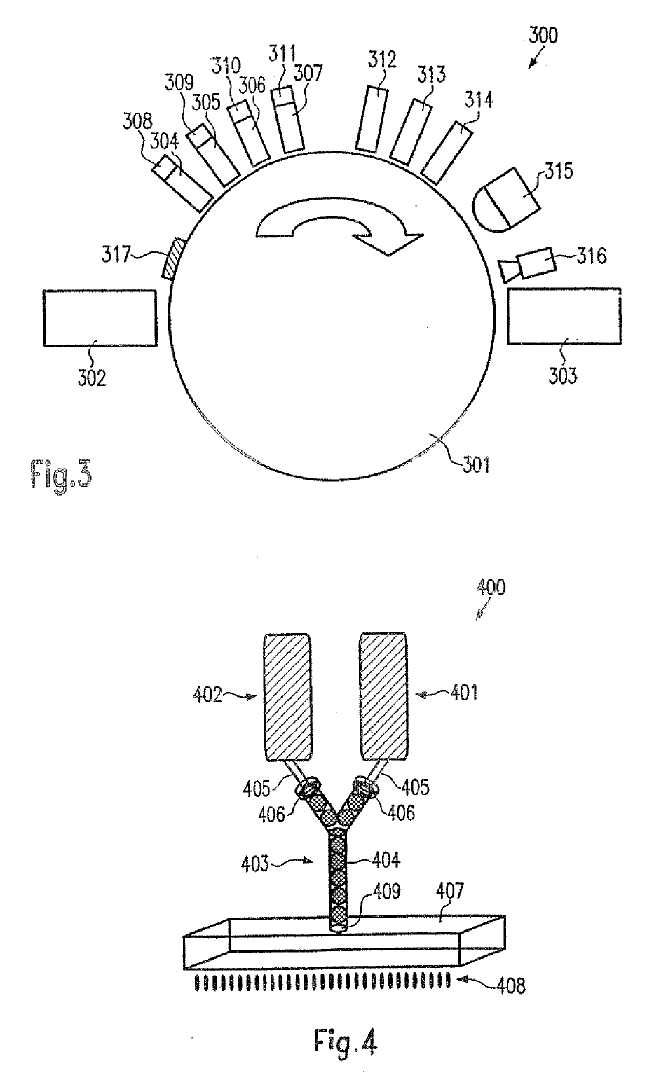

FIG. 3 shows a further schematic embodiment of a system according to the invention;

FIG. 4 shows a further preferred embodiment of a system according to the invention;

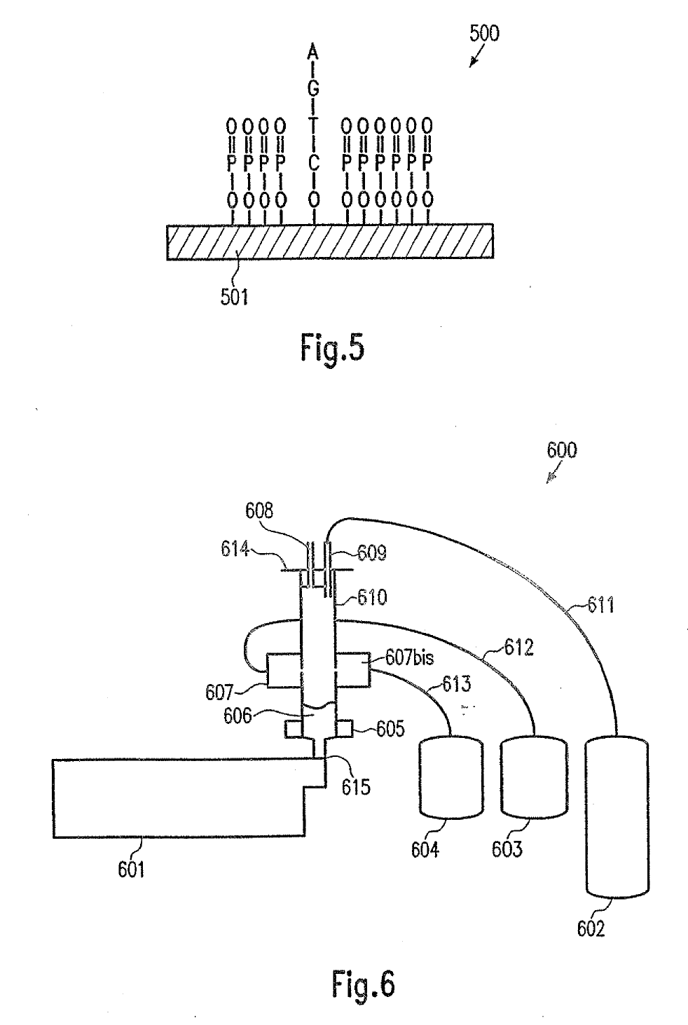

FIG. 5 shows a schematic sectional view of a biochip according to the invention;

FIG. 6 shows a further schematic embodiment of a system according to the invention.

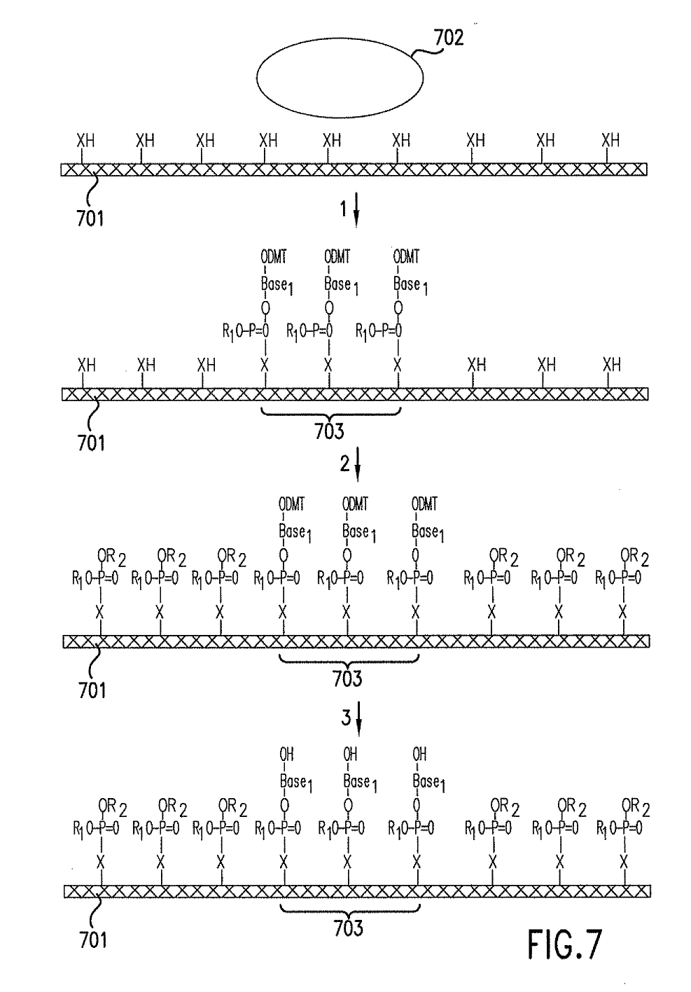

FIG. 7 shows schematically the first reaction step of the method according to the invention.

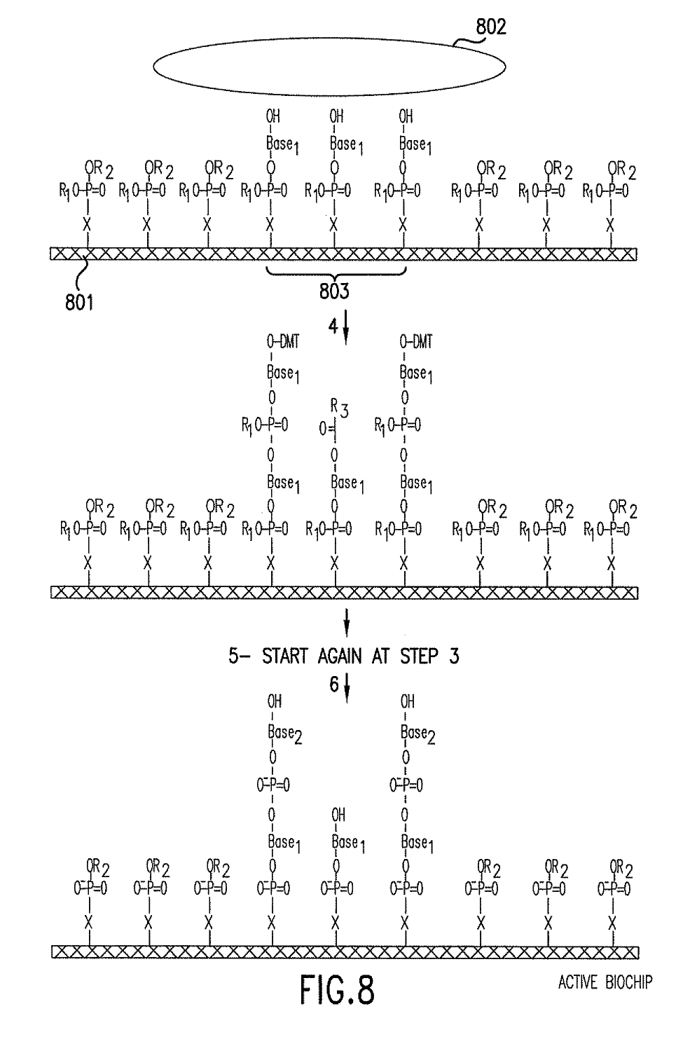

FIG. 8 shows schematically the second and the consecutive reaction step of the method according to the invention.

FIG. 1 schematically shows a preferred embodiment of a system 100 according to the invention. System 100 is arranged within an inert chamber 101 which can be vented with an inert gas such as argon, nitrogen and the like. Within said chamber 101 a substrate 102 is attached to moving means 111. Preferably the substrate 102 is made of an organic polymer such as polyethylene, polypropylene and the like which has activated functional groups on its surface such as, for example, hydroxyl, amine or thiol groups. Moving means 111 allows for moving of substrate 102 in X and Y direction as indicated by the arrow in FIG. 1.

System 100 is designed for the synthesis of, for example, oligonucleotides made of the bases adenine (A), cytosine (C), guanine (G) and thymine (T). For convenience the bases are present as phosphoramidite derivatives. For each base, a reservoir not shown in FIG. 1 is provided. Each reservoir contains a solution of the corresponding base in a solvent. Preferred examples of solvents are glymes, alkylphthalates, alkylsebacates, nitriles like adiponitrile, substituted or non-substituted dialkylethers, benzoic acid derivatives (benzoates and the like) and mixtures thereof. Preferred solvents are high surface tension solvents as mentioned in the foregoing.

Prior to depositing a droplet of the base, each base and a corresponding activator such as, for example, tetrazole (TET) are mixed together in a micro mixing vessel 107, 108, 109 and 110. The volume of a micro mixing vessel is about 4 μl. It is understood, that also smaller or larger volumes can be used in the context of the present invention. The upper limit is about 1 to 5 ml, the lower limit about 10 pl. As a general rule, the volume of the micro mixing vessel comprises the volume of one complete reaction step. The term “complete reaction step” in connection with the volume of the micro-mixing vessel means, that one polymer building block (for example one of the above-mentioned bases) can be applied on every reaction site (“spot”) created in the first reaction step. Therefore the volume may vary according to the specific requirements, the size and the number of substrates to be imprinted.

The microdroplets consist of a pulse of even smaller microdroplets. Each spot is usually constituted by 1 to 100, most preferably by 50 pulses of 1 to 100, most preferred of 20 pL volume for each pulse, that is 1 nl for each spot. A predefined number of spots are forming an array. Several arrays are regularly or irregularly arranged on the substrate and form a biochip. Usually, 96 arrays are arranged on a substrate, but less are more arrays can also be used within the context of the present invention. Therefore the volume of a micro mixing vessel for printing one biochip comprising 96 arrays with 128 spots on each array would be ≅13 μl. If several biochips are to be printed at the same time, the volume has to be adjusted or alternatively the micro mixing vessel has to be refilled after terminating the deposition on the first biochip. It should be noted that the lifetime for the mixture of synthon (base) and activator should not exceed 1 h to avoid degradation. In another preferred embodiment, the “dead volume” of the ink-jet print head constitutes the micro mixing vessel according to the invention.

After mixing the activator and the base in micro mixing vessel 107, 108, 109 and 110, the solution is transferred to an inkjet print head 103, 104, 105 and 106, i.e. one print head for each base. Print head 103, 104, 105 and 106 has a linear arrangement of a plurality of small nozzles. The nozzles are preferentially piezoelectric pumps which are individually addressable so as to generate a microdroplet only from one nozzle out of a plurality of nozzles. The number of the nozzles is either 64 or 128 or in an especially preferred embodiment 256. It is understood that also a different number of nozzles can be used within the context of the present invention. The substrate 102 is then moved to inkjet print head 103 which contains a solution of, for example, A and TET. According to the number of reaction sites to be created onto substrate 102, only one or a preselected number or all of the nozzles will deposit microdroplets onto the surface of the substrate 102. After completion of the reaction, substrate 102 is then transferred to a treatment chamber 112 which comprises a rinsing unit 113, a unit for carrying out the transformation of non-reacted functional surface group into inert species 114 and a deprotection unit 115. In another embodiment of the invention, the substrate 102 constitutes a wall of said treatment chamber 112. The sealing is achieved via pressure of the chamber on the substrate with or without sealing means. Further units not represented in FIG. 1 comprise a further capping unit for carrying out the capping of non-reacted hydroxyl groups of the nucleotides and a final deprotection unit and a oxidation unit for the oxidation of the nucleotide linker phosphorous groups. The final deprotection unit will deprotect (for example with NH₃) the final oligonucleotide to generate if necessary a biologically active oligonucleotide. A typical non limiting sequence of reaction steps is as follows:
1. mixing phosphoramidite+activator, jetting on substrate, washing, oxidation, washing, transformation of unreacted functional surface groups in chemically inert species, washing, deprotection, washing, flushing and drying,
2. mixing second phosphoramidite+activator, jetting on substrate, washing, oxidation, washing, capping, washing, deprotection, washing, flushing and drying, repeating step 2 as often as required,
3. adding NH₃, washing; end.

In another embodiment of the invention, the different bases (i.e. the mixture of the corresponding base and activator) are added subsequently without moving the substrate after each base addition step to treatment section 112. Only after addition of all bases of the same position in the oligonucleotide or after addition of a predetermined number of bases, the substrate is moved to treatment section 112 where it is treated as described above.

It is important that the second microdroplet has a larger diameter than the first microdroplet which defines the reaction site. After the first microdroplet deposition, the non-reacted hydroxyl reactive groups on the surface of substrate 102 are reacted with a agent (“surface capping”), preferably a phosphorous containing reagent for this transformation into a chemically inert moiety during the complete synthesis cycle of the polymer chain (for examples phosphates, perfluoroalkyl or aryl phosphates, C₈-C₂₀ alkylphosphates.

FIG. 2a shows a schematic view onto a print head 200 to be used in the system according to the invention. Print head 200 has a metallic housing 201. At the bottom of the metallic housing 201 a individually addressable nozzle 207 is arranged. The inside of housing 201 contains a chamber 205 where the base mixed with the activator are collected. Chamber 205 is in fluid connection with nozzle 207. In a preferred embodiment, chamber 205 represents the micro mixing vessel according to the invention, where the polymer building block and if necessary the activator are mixed together. Nozzle 207 is addressable via piezo elements 203 which has a piezo connector 204 therefore allowing for the exact definition of the deposition pattern. The print head 200 is further equipped with a fluid supply 206 elements.

FIG. 2b shows an assembly 205 of a plurality of print heads 200. Each print head 200 is connected with another one. The nozzles 207 are linearly arranged to form a nozzle line 202. The number of the nozzles is individually selectable according to the number of print heads 200 used. Preferred are 128 to 256 nozzles. It is understood that also less nozzles for example, 64, 32 and the like can be used within the invention. The print heads 200 are in fluid connection via fluid supply 206 which addresses each print head.

FIG. 3 shows another schematic embodiment of the system 300 according to the invention. System 300 comprises a rotating drum 301, attached thereto is a substrate 317. The substrate is essentially the same as described in the foregoing. Around the drum inkjet print heads 304, 305, 306 and 307 for each corresponding base and activator are arranged. Adjacent to print heads 304, 305, 306 and 307 are micro mixing vessels 308, 309, 310, 311 where the premix of the base to be used and the corresponding activator is formed immediately prior to creating of the microdroplets and jetting the solution onto the substrates.

The treating unit comprises the units 312, 313 and 314 for the rinsing, deprotection and capping reagents. Further units not shown in FIG. 3 comprise but are not limited to oxidation units and a unit for surface capping.

Further, a dryer 315 is arranged around the drum in order to dry the substrate after completion of the reaction. Viewer 316 which may be a computer controlled image viewer which controls the completion of the reaction at every reaction stage. This may be achieved, for example, by using a colorant which is added to the premix of base and activator and which can then be detected visually by usual means inventor in the art, UV/VIS spectroscopy, etc. A preferred class of colorants are azulene dyes.

FIG. 4 shows a schematic sectional view of the system according to the invention with a micro mixing vessel. System 400 has two reservoirs 401 and 402. Reservoir 401 contains the polymer building block, for example a phosphoramidite when an oligonucleotide has to be synthesised (401) and reservoir 402 contains an activator or a solution comprising an activator, for example pyridinium chloride, tetrazole and the like. As already explained in the foregoing, not only one reservoir 401 for the polymer building block and if necessary an activator reservoir 402 is used, but also a plurality of reservoirs 401 for different polymer building blocks and activators respectively are used in a further preferred embodiment of the invention. The polymer building block, i.e. for example a phosphor amidite may be comprised within a solution. Both reservoirs 401 and 402 are connected via tubes 405, for example made of Teflon®, to the micro mixing vessel 403. As already explained under FIG. 1. the micro mixing vessel 403 has a volume of about 40 μl preferably about 5-10 μl. The micro mixing vessel 403 is for example a glass tube in the form of a Y or another suitable form with two inlets, comprising a plurality of frits 404 which minimize the size of a microdroplet at outlet 409. Preferably the frits 404 are made of a chemically inert material such as for example ceramics and the like. The micro mixing vessel 403 is sealed on both inlets which are connected via tubes 405 to the reservoirs 401 and 402 via sealing caps 406. Outlet 409 of the micro mixing vessel 403 is connected to ink-jet head 407. From ink-jet head 407 the microdroplets are applied onto the substrate which is not represented in FIG. 4. The microdroplets not shown in FIG. 4 form micro spots 403 on the surface of the substrate.

FIG. 5 shows a schematic side view of a biochip according to the invention. Biochip 500 comprises a substrate 501 which consists of, for example, polyethylene, polypropylene or mixtures thereof which contain a functional surface comprising oxygen groups. After completion of the reaction cycle as described in the foregoing, on one of the functional groups an oligonucleotide sequence C-T-G-A is attached on the surface of said substrate. A reaction zone was created by the first microdroplet, as described in the foregoing. Around said reaction site the remaining functional hydroxyl groups are transformed in a chemically inert species (surface capping) with a phosphorous containing reagent which is then oxidized to a pentavalent phosphorous to generate a chemically inert phosphate species on the surface of the chip. Substituents at phosphorous are not shown in FIG. 5. Preferred phosphorous substituents comprise perfluorated alkyl, aryl, aralkyl, alkyl groups and the like.

FIG. 6 shows a further schematic sectional view of a system according to the invention with a micro mixing vessel. System 600 comprises an inkjet print head 601 as described in the foregoing. It is understood that the system comprises a plurality of print heads 601 not shown in FIG. 6 whereby each print head is used for a different polymer building block.

For each print head 601, a tank 602 for the wash solvent for example acetonitrile, or another suitable solvent, as for example used for dissolving phosphoramidites, further a tank 603 for the activator and a tank 604 with a solution of the polymer building block are connected to the micro mixing vessel 610. The form and the material of these tanks may be chosen according to the specific requirements as well as their volume. Tanks 602, 603 and 604 are connected via flexible tubes, preferably made of Teflon® or of a similar material to that of micro mixing vessel 610. Micro mixing vessel 610 may be made of, for example, glass, chemically inert plastic material and the like. The specific form may be any form acceptable for the intended purpose. The volume of micro mixing vessel 610 is defined as in the foregoing so that only the exact amount of the mixture between the polymer building block and an activator, mixture 606, is present in micro mixing vessel 610. Tubes 611, 612 and 613 enter via valves 609, 607, 607 bis into micro mixing vessel 610. Additionally, a miniscus system 608 may be placed on the top of micro mixing vessel 610. Micro mixing vessel 610 is sealed via sealing means 614 in order to avoid entry of air. Micro mixing vessel 610 is linked via linking means 615 with inkjet print head 601. Valve 605 is a valve used to control the flow of the mixture 606 to inkjet print head 601. This especially preferred embodiment not only uses micro mixing vessel 610 for the addition of the mixture between the phosphorous building block, for example a phosphor amidite, and an activator but also after reaction for washing the substrate with a wash solvent. Therefore, the set-up can be further simplified and made easier.

FIG. 7 is a schematic representation of the first reaction step of the method according to the invention. The support 701 made of for example polypropylene, polyethylene, polystyrene and the like has functional reactive groups XH on its surface. X is preferably O or N. These functional groups are either obtained during the synthesis of the polymer or after a respective treatment of the polymer like laser treatment, UV radiation, plasma treatment and the like. A first droplet 701 comprising a nucleoside phosphor amidite and if necessary an activator are jetted on the surface of the support 701 by an inkjet print head not represented in FIG. 7. The nucleoside which has a protecting group like DMT or an equivalent protecting group, preferably an orthogonal protecting group, reacts with the reactive functional group XH on the surface of substrate 701. The rest R₁ is any labile group, for example a cyanoethylene group as usually used in phosphoramidite chemistry for the intended purpose. In a second step, the non reacted functional reactive surface groups XH are transformed by the reaction with a phosphorate species into a chemically inert species around the reaction site where the nucleoside phosphoramidite was brought to reaction. R₂ is a non-labile non-removable group, thus rendering the reaction product from step 2 chemically inert. Further, the phosphonate species is resistant to mechanical degradation, as for example scratching and the like and generates areas with a high chemical and mechanical stability between the spots of the reaction sites 703.

After the transformation (“surface capping”) of the reactive functional surface groups in chemically inert species, the orthogonal protecting group DMT is removed by usual techniques essential known to an artisan, thus liberating a free OH group at the 3′ or 5′ end of the nucleoside. The term “base₁” in the context of FIG. 7 means, that the base may be any of the protected bases used in oligonucleotide chemistry like adenine, thymine, cytosine and guanine. The free OH group of the nucleoside is now the starting point for the next synthesis step in the method according to the invention as described below in FIG. 8.

FIG. 8 shows schematically the second reaction step in the method according to the invention. On the substrate 801, which corresponds to the substrate 701 in FIG. 7, a second droplet 802, which has a larger diameter than the first droplet in FIG. 7 (702) comprising a second base, which may the same or different as the base used in FIG. 7, is applied on the reaction site 803 which has been defined in the foregoing first reaction step. After the reaction of the second base, usually used in form as its phosphoramidite, the standard capping of the non reacted hydroxyl group of the first nucleoside is carried out, for example by treatment with anhydrous acetic acid. R₃ is preferably an alkyl group like for example ethylene as in the case of acetic acid. R₁ and R₂ have the same meanings as in FIG. 7. In the next reaction step 5, a deprotection of the DMT protecting groups as explained under FIG. 7 step 3 is carried out. The reaction sequence is carried out until the desired length and nature of the oligonucleotide chain is obtained. The last reaction step 6 is the treatment with aqueous NH3 to yield an active biochip according to the invention.

1-9. (canceled)

10. A method for synthesizing a polymer on a solid substrate comprising the sequential reaction of polymer building blocks comprising reactive groups protected with removable protecting groups comprising the following steps:
a) providing a polymeric surface whereby the polymeric surface comprises functional groups,
b) applying a first microdroplet comprising a first polymer building block on said surface,
c) reacting the first polymer building block with a functional group of the polymeric surface thereby attaching the first polymer building block to the polymeric surface,
d) removing a protecting group of the attached polymer building block
e) applying onto said first microdroplet a second microdroplet comprising a second polymer building block, which is the same or different from the first polymer building block,
f) reacting said second polymer building block with the deprotected reactive group of the first polymer building block, and
g) optionally, sequentially repeating steps d)-f).
wherein after step c) the unreacted functional groups of the polymeric surface are transformed into chemically inert species,
and wherein the diameter of the first microdroplet is smaller than the diameter of the second microdroplet.

11. A method according to claim 10, wherein the first microdroplet has a diameter between 1 μm and 1,000 μm.

12. (canceled)

13. A method according to claim 10, wherein the chemically inert species comprise phosphorous groups.

14. A method according to claim 10, wherein the surface is an integral part of the substrate.

15. A method according to claim 14, wherein the polymeric surface comprises an organic polymer.

16. A method according to claim 15, wherein the reactive groups are selected from OH, NRH whereby R may be H, an alkyl preferably C₁-C₄ alkyl, aralkyl, a cycloalkyl group, SH.

17. A method according to claim 10, wherein the polymer building block is selected from the group consisting of nucleosides, nucleotides, amino acids, peptides and carbohydrates.

18. A method according to claim 10, wherein immediately prior to step b) the activator is mixed with the first polymer building block in a microreservoir.

19-23. (canceled)

24. A method according to claim 15, wherein the organic polymeric substance is polypropylene.
